(19) United States
(12) Patent Application Publication
(10) Pub. No.: US 2017/0202702 A1
(43) Pub. Date: Jul. 20, 2017

(54) Fallopian Tube Occluding Device, Delivery Catheter and Method

(71) Applicant: Bandula Wijay, Friendswood, TX (US)

(72) Inventor: Bandula Wijay, Friendswood, TX (US)

(21) Appl. No.: 14/997,077

(22) Filed: Jan. 15, 2016

(51) Int. Cl. A61F 6/22

(52) U.S. Cl. CPC A61F 6/22

(57) ABSTRACT

A permanent acute occlusion implantable device for sterilization of human female and method are described for immediate occlusion of the fallopian tubes of the human female, wherein an structure having an a shape to the normal anatomy of the ostium of the fallopian tube device consisting of a sealing segment is placed to seal the ostium of the fallopian tube. The sealing segment of the device is encased in an elastomeric material to provide the sealing action once the device is delivered into the ostium of the fallopian tubes. The occlusion device is held in place due to the spring action of the intermediate connecting segment which in turn is connected to an expanding anchoring segment. The occlusion device is delivered to the final location via a delivery sheath which is threaded through a hysteroscope.

### FIELD OF THE INVENTION

The present invention relates to an endo-luminal occluding device, which when implanted into the fallopian tubes, facilitates immediate occlusion of the lumen and sterilization of human female. By this means, immediate blocking of the fallopian tubes will prevent sperm migration into and through the fallopian tube or the egg migrating from the ovary to the uterus and thereby preventing the fertilization of the ovum and hence will provide means of immediate female sterilization.

### BACKGROUND OF THE INVENTION

The present invention deals with a method and device that will occlude the body lumen immediately and does not depend of tissue growth to cause the occlusion of the body lumen. Occlusion of body lumens for various medical procedures as well as for female sterilization is a very common practice and is performed frequently throughout the world except in most cases the procedure depends of tissue growth caused by tissue injury to affect closure, which takes time. Often additional tests are conducted at a later date, with additional expenditure, to assure, a closed lumen. Additionally, the most common female sterilization method is fallopian tube ligation, a procedure that utilizes a trans-abdominal approach for the occlusion, or tying, of the fallopian tubes, which is surgical, and expensive. Despite its worldwide use, tubal ligation via the trans-abdominal approach is associated with substantial trauma, discomfort, hospital stays, and complications, such as bleeding, infection, reactions to general anesthetic, and bowel perforation. The trans-abdominal approach involves surgery, and is not readily available to many women throughout the world. Even though local anesthetic is considered an option for the trans-abdominal approach to tubal ligation, almost all of these sterilization procedures are performed under general or spinal anesthesia. In addition, the trans-abdominal approach to tubal ligation requires incisions that invade the peritoneal cavity, thereby raising the risk of injury to intra-abdominal structures.

In order to avoid the problems associated with trans-abdominal tubal ligation procedures, various trans-cervical approaches to tubal sterilization have been proposed. The trans-cervical approach to sterilization involves the insertion of a catheter or sterilization device directly into the fallopian tubes via the reproductive tract, eliminating the need for general anesthetic and abdominal incisions. Initial trans-cervical approaches to tubal sterilization involved radiofrequency, chemical or heat induced scarring, or liquid silicone injections. However, these approaches have all failed due to safety and efficacy concerns. Chemical scarring agents, such as quinacrine, iodine, and methylcyanoacrylate, require repeated applications and have problems concerning biocompatibility. Thermal blocking procedures, which induce the formation of scar tissue within the fallopian tubes, have high failure rates and major complications such as uterine bleeding and bowel perforation. Electrocautery methods, which employ an electric current to induce scar tissue within the fallopian tubes, are also unsatisfactory because they do nor scar a sufficient amount of tissue and because they can burn surrounding organs, particularly the bowel.

Current trans-cervical methods involve occluding the fallopian tubes by implanting a small occluding device. The occluding devices in the prior art are usually in the form of a cylindrical plug or a coil. For instance, Loy in U.S. Pat. No. 6,357,443 describes a removable fallopian tube plug consisting of a tubular (cylindrical) elongate member with a number of fingerlike protrusions that extend radially outwards creating a barrier and thereby occluding the fallopian tubes. Additionally, Saadat et al. in U.S. Pat. No. 5,935,137 describe a fallopian tube occluding device for female sterilization which is a plastic, rubber, or metal elongate hollow tubular (cylindrical) structure with ribs that are either coated with copper or are interlaced with copper rings. The hollow portion of this device has a valve, or seals with a hydrogel, after the device is implanted into the fallopian tubes.

Coils, which have a helical outer surface and which assume a bent shape when released from the delivery catheter system, are also used to occlude the fallopian tubes. For example, Ton et al. in U.S. Pat. Nos. 5,601,600 and 5,746,769 describe the use of a coil to occlude the fallopian tubes. The device consists of polyethylene terephthalate (PET) fibers wrapped around a stainless steel core that is surrounded by 24 or more coils of nickel-titanium alloy. After the device is deployed within the fallopian tubes, the PET fibers induce the tubal epithelium to undergo fibrosis, which results in tubal occlusion. The device also relaxes to its natural bent shape once it is deployed in the fallopian tube. The tubal occlusion process from these devices takes about three months to complete and must be confirmed via a hysterosalpingogram.

U.S. Pat. No. 7,846,160 teaches the use of an exterior screw thread to advance a lumen plug by rotation In FIG. 3. In FIG. 4 barbs are held under a sheath such that when the sheath is retracted, the barbs spring out and point proximally to prevent removal.

US 2007/0227544A1 describes an intra-corporeal occluding device having elements with non-traumatic ends to occlude lumens for either occlusion or for the delivery of drugs to the lumen.

U.S. Pat. No. 8,695,605 B2 describes a fallopian tube occlusion device having a “U” shaped element made from self-expanding nitinol metal having an outer material encapsulating the said “U” shaped element.

U.S. Pat. No. 5,656,036 describes a lumen occlusion device providing permanent occlusion consisting a flexible radially expandable metallic stent connected to a flexible closure member having a generally tubular shaped cross sectional configuration.

US 2008/0178890 A1 describes a device for occluding the fallopian tube using a cap portion removable connected to an implanted portion. The cap portion consists of a substantial impermeable membrane and is mean to occlude the ostium to the fallopian tube.

Most of these devices eventually become dislodged or have been found to be only moderately effective in preventing pregnancy. Most have incidents of migration, including migration of the device into the abdominal cavity resulting in serious infections. Devices invented and developed in the past needed post implantation confirmation tests to confirm the efficacy of occlusion causing additional pain and expenses to the patients. The occlusion device of the present invention fulfills these unfilled needs.

### SUMMARY OF THE INVENTION

The present invention consists of an acute and permanent occlusion implantable device (Occlusion Device), for permanent sterilization of human female and delivery method thereof for immediate occlusion of the fallopian tubes of the human female to prevent future conception, thereby sterilizing the subject. The implant, an acute occlusion device, consists of a sealing means to seal the opening (ostium) of the fallopian tube, an anchoring means to anchor the device within the fallopian tube and intermediate connecting means to connect the sealing means and the anchoring means such that the sealing means is pulled towards the anchoring means to provide a force, to pull the sealing means tight against the opening of the fallopian tube, thereby completely sealing the opening of the fallopian tube. The aforementioned device will prevent the sperm traveling up into the fallopian tubes and the egg traveling down the fallopian tube into the uterus due to this seal and hence fertilization of the egg. The sealing means is a self-expanding metallic or non-metallic structure lined with an appropriate sealing membrane to provide a seal. The anchoring means and the connecting means are also self-expanding metallic or non-metallic structures that will expand on delivery to its designed dimensions to hug the inner walls of the fallopian tubes. The device can also be designed to work as a balloon expandable structure that will be expanded by an inflatable balloon such that the device is expanded to a predetermined size after balloon inflation.

The occlusion device described in the present invention is permanently placed inside a body lumen for occlusion of the body lumen. The device is delivered using a sheath from within which the device is delivered to the appropriate location in the fallopian tube under visual or fluoroscopic guidance and by withdrawing the sheath, thereby delivering the device into the desired location such as the ostium of the fallopian tube. The delivery of the device is also possible by crimping the device on to an inflatable balloon where the occlusion device is crimped onto a balloon. The delivery catheter is threaded into the appropriate location either through a sheath or through a hysteroscope and the balloon is inflated to deliver the occlusion device into place causing immediate occlusion of the lumen.

The sealing means consists of a conical structure, which can be made out of slotted a tube or from a wire form frame. When the conical structure is made of wire form, such wire form can be either made from braided tube or can be wire formed and bent into a shape to maintain the conical nature of the sealing means. When it is made from a slotted tube, this small narrow metal frame is made from a metallic tube of small diameter, such as 1.5 mm tube having a wall thickness of 0.13-0.15 mm by cutting appropriate slots, or openings, on its surface, so that expanding of the slotted tube due to its shape memory yields a structure that is several times larger than its original diameter and continues to remain at its final expanded conical shape. Similar structure can be made from molded polymer such as polypropylene or nylon having appropriate memory characteristics. This sealing means has a general conical shape, with the cone apex directed towards the opening of the fallopian tube. The cone is covered or lined with a sealing material such as a suitable elastomeric material. The materials known for their sealing properties, such as polyurethane or silicone would be ideal for use for this purpose. The sealing material is made to line the distal (outside) surface of the cone and ends up in a cul-de-sac, so that the sealing means consists of a complete obstruction of the fallopian tube canal. The material of the conical structure will undergo deformation beyond its yield point on delivery and therefore will remain at or near its expanded condition. Often, a slightly higher expanded diameter may be required to achieve the final diameter needed for the expanded conical structure for a given application. The initial diameter of the slotted tube or wire form is substantially small (less than 2 mm) so that the initial profile of the slotted tube and the entire device is small enough to thread through a delivery device catheter or a hysteroscope depending on the purpose of its use and where the occlusion device is implanted. The slots or openings on the surface of the slotted tube can be lengthwise or at an angle to its longitudinal axis. The number of slots around its diameter also depends on the surface available on the slotted tube for providing sufficient slots. The slots can be arranged in many different configurations having the general sinusoidal or triangular undulations or any other shape that would make the slotted tube flexible. The width and the length of the slots are determined by the initial diameter, hence the surface area, of the slotted tube and the ratio of the expanded and the non expanded diameter of the slotted tube. A larger number of undulations are typically provided when the structure is expanded to a larger diameter and vice versa.

The anchoring means is also self-expanding wire form or metal or plastic slotted tube. The anchoring means can be made by cutting slots into a tube as previously described, or from a braided tube or from a wire form, which has been heat set to expand to a predetermined size and shape. The slots can be arranged in many different configurations having the general sinusoidal or triangular undulations or any other shape that would make the slotted tube flexible. The width and the length of the slots are determined by the initial diameter, hence the surface area, of the slotted tube and the ratio of the expanded and the non-expanded diameter of the slotted tube. The shape is typically conical in nature to match the diverging anatomical nature of the fallopian tube. The geometrical nature of the anchoring means also prevents the said device from migrating to the uterus or out of the fallopian tubes.

The afore mentioned connecting means is also a self-expanding wire form, braid or metal or plastic slotted tube. The connecting means of the device is designed such that on expansion, the connecting means pulls the sealing means towards the anchoring means. The design of the slots and or the braiding is to create afore mentioned pull force once the device is delivered. The sealing membrane which is attached to the conical structure of the sealing means generally ends in a cul-de-sac within the connecting means.

The acute occlusion implanted device is mounted internally into a sheath, which is part of a delivery catheter. The delivery catheter is threaded through the hysteroscope which is inserted through the cervix into the uterus. The said device is released from the sheath by retracting the sheath. The device is then delivered to the ostium of the fallopian tubes or into similar body lumens for permanent implantation and thereby occluding the said lumen or the fallopian tube permanently. A similar result can be achieved (not shown) by using a delivery balloon and delivering the acute implanted occlusion device by inflating the balloon and delivering it to the ostium of the fallopian tube. The delivery catheter will have a relatively small shaft such as 2-5 French and will have sufficient stiffness and flexibility that allows optimal push-ability and track-ability characteristics to track through the hysteroscope and deliver the device into the fallopian tube.

The sealing conical structure, for sealing the ostium of the fallopian tube, consisting of an expanding slotted tube, braid or wire form, is encased in a bio-compatible expandable material, such as silicone, polyurethane or any other soft, expandable polymeric or biological material that can be used to encase the conical structure of the occluding device. The encasing material shall be well bonded to the metallic struts of the slotted tube and the struts of its cone or wire forms and shall not separate from the struts during expansion. The design of the struts of the conical sealing section shall be in such manner to provide a smooth sealing surface.

The anchoring means of the occlusion device is similarly made from either a slotted tube or molded plastic or a wire form which self-expands to its predetermined shape on delivery. The slots can be arranged in many different configurations having the general sinusoidal or triangular undulations or any other shape that would make the slotted tube flexible. The width and the length of the slots are determined by the initial diameter, hence the surface area, of the slotted tube and the ratio of the expanded and the non-expanded diameter of the slotted tube. The slots can be so designed that on expansion, the undulations created thereby are generally projecting diametrically outwards and shall have a fish scale appearance and helps to anchor the anchoring means to the inner surface of the expanding fallopian tube.

The connecting means section is also made of a slotted tube or wire braid. This portion stays in the neck region of the fallopian tube and shall have a smaller profile giving it an hour-glass shape and hence fitting the general anatomy of the fallopian tube ostium. This portion of the occlusion device shall also be flexible, and expands to its predetermined diameter upon delivery. The strut design shall be such that after placement, it provides a “pull” force to pull the sealing cone towards the anchoring means. The sealing member which is attached to the conical structure ends in a cul-de-sac within and inside the connecting means section.

The acute occlusion implantable device mentioned here consisting three segments as previously described and can be made as one contiguous assembly, or hereafter referred to as the scaffold. When the scaffold is made as a slotted tube, it can be made from one single tube, slotted by laser cutting and expanding to the desired diameter and shape or by molding the scaffold from a suitable plastic such as polypropylene or nylon. If it is made from a wire, it can be made from wire braided and heat set to the desired shape or from wire form shaped into the desired size and shape and by heat treating to set the desired shape. The slotted tube design generally has several undulating or sinusoidal segments relatively perpendicular to the axis that are connected with flexible connecting strut members. The undulating segments can have different amplitudes and pitches and shapes in order to provide the function of afore mentioned segments and can be at an angle to the longitudinal axis, although they are generally perpendicular. The connecting strut members can be straight, parabolic or sinusoidal and may have more than one tangent thereby creating a curved strut. The connecting struts can be very short (less than 0.5 mm) or long (more than 0.5 mm). The preferred embodiment will consist of one to three-four undulating rings in the slotted tube in the sealing means segment, two-three undulating segments in the connecting region and 3-5 undulating segments in the anchoring region.

The acute occlusion implantable device scaffold can be made from Nickel-Titanium or commonly known as Nitinol alloy. Or it can be made by braiding a tube using Nitinol wire followed by heat setting it to the desired shape or simply from Nitinol wire bent to the desired shape and heat set thereafter to the desired shape. The Nitinol scaffold so formed by a laser cut slotted tube or formed by bending wire or braided wire shall be coated first with Titanium by sputtering methods to coat the surface of the scaffold with a few microns thick layer or is coated with such polymeric materials like polyurethane or other bio-compatible materials. The sealing member is affixed to the scaffold by encasing the membrane on to the struts in the sealing means segment by dipping or gluing the sealing member on to the structure. The sealing member terminates in a cul-de-sac with a completely closed pouch, which shall generally end in the connecting segment of the occlusion device.

Once the occluding device is properly delivered, a hermetic seal is made between the uterus and the fallopian tube preventing fluid passage in both directions, thereby preventing the egg and the sperm meeting each other to prevent conception. The cone portion of the occlusion device is pulled towards the ostium of the fallopian tube by the spring force exerted by the connecting segment and therefore keeps the cone pressed firmly to the ostium providing a reliable hermetic seal. The space between the two undulating segments in the connecting segment is connected by a few struts (typically three to four). These struts are designed in a spiral configuration to act as springs providing the special benefit in providing the spring force needed to pull the cone towards the ostium. The undulating segments of the anchoring segment generally are of shorter width so that on expansion the free ends of the undulations will project outwards and therefore helps in anchoring the device in to the lumen of the fallopian tube. Additionally the inner wall tissue will also prolapse through the opening in the scaffold affixing the occlusion device in to the lumen.

The encasing materials, such as silicone and polyurethanes usually have elastic properties. As such when the slotted tube scaffold is expanded, there is some degree of recoil (back), which may cause an improper hermetic seal. Therefore the slotted tube or the wire form also referred to as the scaffold should be designed slightly larger than the final diameter for the application and the sealing material is affixed onto the scaffold in its expanded condition. The occlusion device has a generally an hour glass shape. A shape similar to the anatomical detail of the typical uterus and fallopian tube transition. The hour glass shape of the occlusion device fits well to the anatomy and the shape prevents the occlusion device from migrating in either direction and the features of the present invention provide a permanent hermetic seal between the uterus and the fallopian tube.

The above described device is an acute and permanent occlusion device, which is made from a scaffold fully or partially encased with an expanding, yet impervious membrane. Upon delivery and the occluding device is anchored in the ostium and provides an occluding “wall” completely closing the ostium and preventing the flow of fluids across the device. As such, this device and various modification of it will provide permanent occlusion of a fallopian tube when properly implanted in a body lumen. The advantage of the present invention is that immediate hermetic seal and a complete and immediate occlusion of the ostium are achieved by the proper placement of the occluding device. This device does not depend on tissue growth within the fallopian tube to close the lumen, which not only takes time but in some instances not very reliable and therefore does not require a test after a few weeks to confirm for the efficacy of occlusion. The advantage of the present inventions is that the hourglass shape prevents the device migrating out of the implanted location as it mimics the natural anatomy of the fallopian ostium and stays locked into its place. The device is flexible and due to its natural shape, it causes minimal spasm and prevents in severe abdominal pain and discomfort. This property of the proposed device also helps in minimizing migration which is caused by the spasm of the fallopian tube. Therefore the device in the present invention to occlude the ostium of the fallopian tube is unique both in its configuration and its interaction with the anatomy of the uterus of the human female.

### BRIEF DESCRIPTION OF THE DRAWINGS

FIG. 1 shows the typical anatomy of the ostium and the fallopian tube of the human female.

FIG. 2 shows the fallopian tube occlusion device in the fallopian tube, occluding the fallopian tube ostium.

FIG. 3 shows the detailed design of the occlusion device.

FIG. 4 shows the general shape of the occlusion device.

FIG. 5 shows the detailed design of the structure and undulations of a typical occlusion device.

FIG. 6 shows the occlusion device within the delivery sheath.

### DETAILED DESCRIPTION OF THE PREFERRED EMBODIMENT

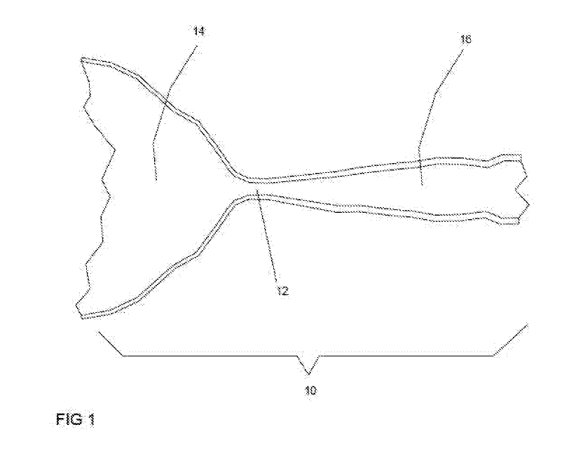

FIG. 1 shows the typical anatomy 10 of the entrance to the fallopian tube (16) from the uterus (14). It consists of a conical entrance having a large angular portion in the shape of a cone and a narrow lumen (12) that gradually expands to an irregular yet another conical structure that ends in irregular diverging funnel type anatomy. The egg from the ovary travels down this tube (16) to the uterus (14) and is fertilized when in contact with the sperm.

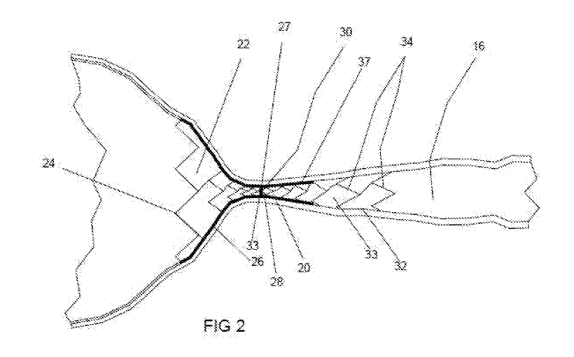

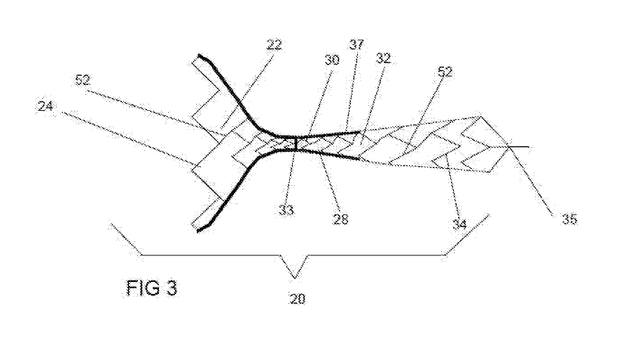

FIG. 2 shows the proposed acute and permanent occlusion device that permanently hermetically seals the fallopian tube so that the sperm and the egg do not get in contact with each other and hence causes permanent sterilization of the human female. The occlusion device (20) consists of three segments. Segment (22) is the sealing segment that abuts with the ostium of the fallopian tube completely sealing the entrance to the fallopian tube, segment (28) that is smaller in diameter and lies within the proximal fallopian tube (16), which is a narrow tubular portion which having a gradually increasing diameter, and the diverging segment (32) that abuts the said diverging portion of the fallopian tube (16). The general nature of the occlusion device (20) is such that it becomes the shape that is similar in nature to the anatomy of the fallopian tube when delivered and implanted into the fallopian tube. The occlusion device, due to its form, shape and stiffness will therefore mold the fallopian tube to match its form and shape. This nature of the occlusion device and the elastomeric sealing member (26) therefore will completely seal the fallopian tube. The segment (22) consists of a structure (24), which can be achieved either by a wire form (forming wire to a desired shape such as a braid or knit) or by laser cutting a structural pattern into a tube and subsequently expanding the tube structure into the desired conical shape. This structure (24) in segment (22) is lined with an elastomeric or non-elastomeric polymer membrane (26). The membrane (26) abuts to the tissue in the entrance to the fallopian tube resulting in a complete seal. The membrane (26) is shaped to end in a cul-de-sac (27) in the proximal fallopian tube and continue as a funnel (37) into the diverging portion on the occlusion device (32) in the distal fallopian tube. The connecting segment (28) is relatively short (4-5 mm) and lies in the proximal portion of the fallopian tube. The segment (28) is designed to have a spring force that causes to pull the segment (22) towards the ostium of the fallopian tube and resulting in a hermetic seal. The segment (28) can also be made from a wire form or by laser cutting a specific pattern into a tube and expanding it to the desired size. The segment (28) may have struts (30) that are designed in such a manner to cause and act as a tension spring in this segment. A portion of the sealing element (26) continues within the segment (28) forming a partition wall (33) and continues to form a funnel (37) into the segment (32). The segment (28) continues as segment (32), where the structure has and diverging shape mimicking the anatomy of the fallopian tube (16). The segment (32) is also can be made from either a wire form, wire braid or by using a laser cutting a pattern to a tubular structure. The shape of the wire form or the laser cut tubular structure shall have a design that allows a flexible structure that can be compressed into a smaller diameter to enable the occlusion device (20) to be placed inside a delivery tube (40).

The three aforementioned segments, (22), (28) and (32), of the occlusion device is contiguous and are made from a single piece of tube by laser cutting a pattern on to it or is a wire form, formed by braiding or knitting wire and in either case by heat treating to set the shape. In braiding, the pattern of the braid can be either uniform, having similar cell structure throughout or can be varied with different cell structures. The “cell” (33) is defined as the openings in the wall of the braided structure of the wire braid. The larger diameters of the occlusion device may consist of cell structures that have larger openings while the smaller diameters of the occlusion device may contain smaller openings.

The distal end of the structure can be designed to converge into a point and the end can be secured and mated by using an adhesive or by welding or simply twisting the ends of the structure. In case the occlusion device is made from a wire braid, the distal end wires are twisted and secured to prevent unraveling of the wire ends as shown by (35) in FIG. 3.

FIG. 3 shows a detail of the occlusion device (20), which can be made by laser cutting a specific shape into a tube made from nickel-titanium or stainless steel alloy, such as Nitinol or Cobalt-Chromium alloy tubes and expanding and heat setting the desired shape.

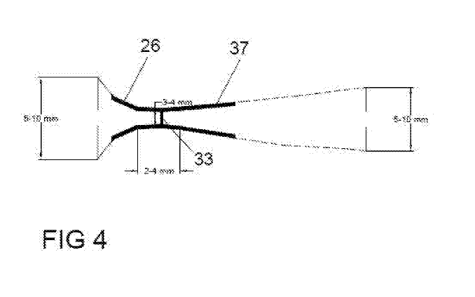

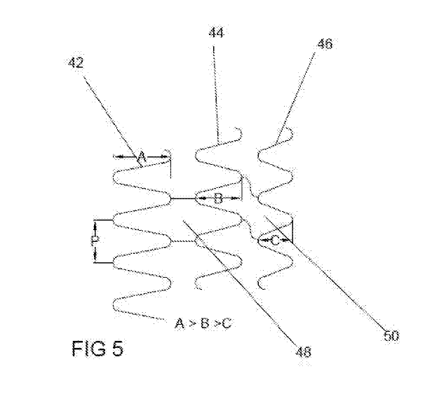

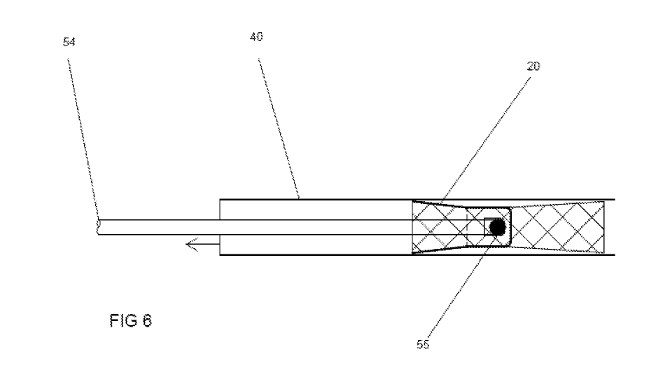

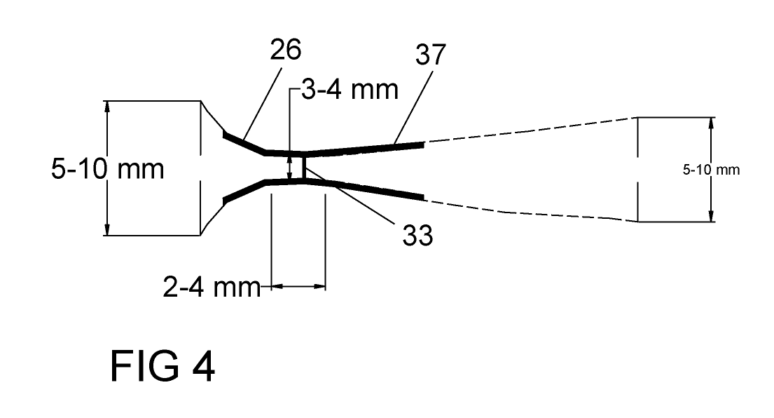

The desired shape of the occlusion device is as shown in FIG. 4. As disused before the segment occupying the ostium to the fallopian tube has a conical converging region followed by a relatively small diameter connecting segment, which gradually expands into a diverging segment having a larger diameter at its further most distal end. As an example the mouth of the structure shall have a diameter of 5-10 mm and the connecting region shall have a waist of about 3 mm and the structure diverges gradually to a final diameter of 5-10 mm. The slots, defined as the shapes cut into the slotted tube shall be a series of relatively perpendicular sinusoidal undulations (24) perpendicular to the longitudinal axis connected by flexible connections (52). The sinusoidal undulations may have varying amplitudes “A” and varying pitches “P”. The undulations are relatively perpendicular to the longitudinal axis; however the present invention shall also include such undulations that are angular to the longitudinal axis at angles other than 90 degrees.

FIG. 5 describes these amplitude variations. For example the sinusoidal segment (42) has amplitude of “A” while the sinusoidal undulations (44) and (46) have amplitudes “B” and “C” respectively. The amplitude of A is greater than that of B and amplitude B is greater than that of C. Similarly the pitch in each undulation can also be different depending on the degree of “open area” (48) desired in the final structure of the occlusion device (20). The open area is defined as the area that is without any metal and where the tissue can prolapse into the structure (20) which in turn helps to anchor the occlusion device. This design enables the slotted tube to be expanded varying diameters without causing undue stress in the structure while providing sufficient open areas for anchoring and additionally once it is expanded it would have minimum recoil when the occluder (20) is delivered into place. The structure of afore mentioned has openings (48) when the sinusoidal undulations are laser cut into the tube. When more openings are designed into the slotted tube stent, either as straight openings (48) or as openings with curvatures (50), less metal content will be left in the structure and hence will have less radial strength but will be more flexible. When fewer openings (48) or (50) are designed into the structure, the structure will be more rigid and will have more radial strength but will be less flexible. This balance of stiffness and flexibility is achieved by making the structure with varying amplitudes and pitches as previously discussed and by designing into the structure appropriate diameter (thickness and width) of the undulations and the connecting struts of the structure.

FIG. 6 shows the occlusion device (20) inside the sheath (40). The occlusion device (20) is placed inside the sheath (40) having a small diameter of around 2 mm, so that it can be threaded through the hysteroscope or other endoscopic or laparoscopic device. In order to thread the sheath (40) through the hysteroscope, the occlusion device needs to be flexible, and therefore the exact design of the occlusion device will be affected by its design and needs to consider the number and type of slots or openings. The occlusion device (20) is delivered into the ostium of the fallopian tube by threading the distal end of the sheath 2-3 cm inside the fallopian tube and gradually withdrawing the sheath (40). The wire member (54) which ends distally in a ball (55) is fixed in space so that when the sheath is withdrawn gradually as in the direction shown by the arrow in FIG. 6, the occlusion member stays put and is held in place by the wire member (54) and does not move backwards with the sheath (40). The sheath (40) is withdrawn until the entire occlusion device is out of the sheath (40). Once occlusion device (20) is deployed, the sheath and the wire member (54) are both pushed forward so as to seat the occlusion device firmly into the ostium of the fallopian tube thereby completely and immediately causing a hermetic seal of the fallopian tube.

I claim:

1. A female sterilization device for hermetically sealing a Fallopian tube, comprising:
a flexible body having proximal and distal ends larger than a connecting segment therebetween and configured for placement of said connecting segment adjacent an entrance to a Fallopian tube such that said proximal segment supporting a sealing surface has said sealing surface positioned against a uterus wall adjacent said entrance for sealing thereof.

2. The device of claim 1, wherein:
said body has a tubular shape.

3. The device of claim 2, wherein:
said body has multiple openings.

4. The device of claim 3, wherein:
said openings are formed using a braid pattern of wire or connected sinusoidal patterns of wire perpendicular or skewed to a longitudinal axis of said flexible body or cuts in sheet formed into a tubular shape.

5. The device of claim 1, wherein:
said distal end exerting a force on said proximal end through said connecting segment in a direction that brings said sealing surface against the uterus wall.

6. The device of claim 1, wherein:
said proximal end comprises a conforming shape to the uterus wall with said sealing surface mounted to a distal side of said proximal end for placement against said uterus wall.

7. The device of claim 1, wherein:
said proximal end comprises a tubular shape defining a passage and comprising a plurality of wall openings covered by said sealing surface to seal against the uterus wall, said sealing surface continuing across said passage to close said passage.

8. The device of claim 7, wherein:
said distal end and connecting segment continue said passage with said sealing surface closing said passage in said connecting segment.

9. The device of claim 8, wherein:
said distal end and connecting segments comprise openings that are formed using a braid pattern of wire or connected sinusoidal patterns of wire perpendicular or skewed to a longitudinal axis of said flexible body or cuts in sheet formed into a tubular shape.

10. The device of claim 1, wherein:
said flexible body comprises a profile of an hourglass.

11. The device of claim 4, wherein:
said sinusoidal patterns comprise varying amplitude or pitch.

12. The device of claim 9, wherein:
said sinusoidal patterns comprise varying amplitude or pitch.

13. The device of claim 1, wherein:
said flexible body is coated with titanium.

14. The device of claim 1, wherein:
said flexible body comprises a biocompatible coating.

15. The device of claim 1, further comprising:
a sheath to reduce the dimension of said flexible body for positioning in the Fallopian tube and a wire member to advance said flexible body to the Fallopian tube;
said sheath selectively movable with respect to said flexible body to release potential energy in said flexible body for fixation while said wire member prevents movement of said flexible body with respect to said sheath.

16. The device of claim 15, wherein:
said wire member pushes said flexible body against the uterus wall as said sheath is removed from said flexible body to insure contact of said sealing surface to the uterus wall.

17. The device of claim 15, wherein:
said removal of said sheath releases stored energy in said flexible body resulting in a net axial force on said sealing surface of said proximal end toward the uterus wall.

18. The device of claim 15, wherein:
said sealing surface comprises an elastomeric or non-elastomeric polymer membrane.

19. The device of claim 1, wherein:
said sealing surface comprises an elastomeric or non-elastomeric polymer membrane.

20. The device of claim 15, wherein:
removal of said sheath allows said flexible body to radially expand for immediate occlusion of the Fallopian tube.

21. The device of claim 4, wherein:
said wire or sheet is metallic or non-metallic.

22. The device of claim 9, wherein:
said wire or sheet is metallic or non-metallic.

23. The device of claim 4, wherein:
said wire or sheet comprises nickel-titanium or stainless steel.

24. The device of claim 9, wherein:
said wire or sheet comprises nickel-titanium or stainless steel.

25. The device of claim 9, wherein:
said sealing surface continues past said connecting segment on an outer surface of said distal end, said proximal end pulling said sealing surface on said distal end against a wall of the Fallopian tube for sealing therewith.
